(19) United States
(12) Patent Application Publication
(10) Pub. No.: US 2007/0255101 A1
(43) Pub. Date: Nov. 1, 2007

(54) Endoscope with Protective Sleeve

(75) Inventor: Yakov Bar-Or, Vardia (IL)

Correspondence Address: BRUCE E. LILLING;LILLING & LILLING PLLC, P.O. BOX 560, GOLDEN BRIDGE, NY, 10526, US

(73) Assignee: SIGHTLINE TECHNOLOGIES LTD., Haifa (IL)

(21) Appl. No.: 11/276,440

(22) Filed: Feb. 28, 2006

Related U.S. Application Data
(60) Provisional application No. 60/658,564, filed on Mar. 4, 2005.

(51) Int. Cl. A61B 1/273

(52) U.S. Cl. 600/124

(57) ABSTRACT

A gastroscopic apparatus for inspecting a body channel is described. The apparatus comprises an insertion tube, an operating handle, a dispenser, which is detachably coupled to the insertion tube, a covering sleeve, which is retained within the dispenser, and a mouthpiece, which is provided with a means for arresting the dispenser within the mouthpiece. Upon arresting the dispenser within the mouthpiece and advancement of the insertion tube within the body channel, the covering sleeve extends from the dispenser and deploys around the insertion tube to protect from contamination that portion of the insertion tube, which had passed the dispenser.

### FIELD OF THE INVENTION

The present invention relates generally to the field of endoscopy and specifically to flexible endoscopes used for medical examination of an intestinal system during which an insertion tube provided with an optical head is put through the mouth and down the esophagus. An example of possible implementation of the present invention could be a gastroscope for viewing the stomach or duodenum and for removing tissues from these organs. It should be kept in mind, however that the present invention is not limited to gastroscopes and can be implemented in any other endoscopes employed for medical examination of the intestinal system, e.g. in duodenoscopes, sigmoidoscopes, etc.

### BACKGROUND OF THE INVENTION

The main components of a modern gastroscopic apparatus comprise a flexible insertion tube fitted at its distal end with an optical head, an operating handle for manipulating the insertion tube during its advancement within the body organ and a system control unit provided with a source of compressed air, water and vacuum to be supplied to the body organ during the gastroscopic procedure. The insertion tube is introduced within a patient's mouth through a dedicated mouthpiece held by the patient's teeth. The mouthpiece guides the insertion tube during the gastroscopic procedure.

Flexible endoscopes in general and gastroscopes in particular are notoriously difficult to clean and disinfect thoroughly, leading to problems of cross-contamination between patients and between patients and staff. These problems can be partially avoided by covering the endoscope with a single-use sleeve, which is discarded after use. The use of a disposable sleeve (also referred to as a sheath) to cover an endoscope is well known in the art.

Endoscopes commonly have working channels, running from a proximal port outside the body to a distal port at the distal end of the endoscope. When the distal end of the endoscope is inserted into the body organ, the working channel may be used to pass a surgical instrument through to the distal end of the endoscope in order to perform a surgical procedure, such as a biopsy.

Instruments that are used in this manner become contaminated with biological matter from inside the patient's body. As the instrument is withdrawn from the body, it spreads the contamination to the interior of the working channel and to the proximal port of the endoscope and eventually to the operator's hands.

Therefore it would be very beneficial to prevent spread of contamination originating from the endoscope itself or from the surgical instrument

Ouchi (US Publication 2003/0097043) describes a cover for preventing contamination of an operating portion of an endoscope. The cover is formed in a bag-like shape for enveloping a total of the operating portion. The cover can prevent exposure to contamination during the endoscopic procedure when the insertion tube is inside the patient. However, during the procedure and in the end of the procedure, the insertion tube is withdrawn from the patient and, since it remains uncovered, the spread of contamination originating from the insertion tube would not be prevented.

Chinese patent CN 1,486,666 describes an endoscope system fitted with a disposable sheath, which at least at its distal end is made of transparent material. The sheath seals all the inserted portion of the endoscope. Various hard-to-clean, open-ended channels of the endoscope, including working channel for surgical instrument, are arranged outside the insertion tube and are disposed of together with the sheath after single use.

Still further methods for sheathing an endoscope for protecting it from contamination are described, for example, in Silverstein (U.S. Pat. No. 4,646,722) and Sidall (U.S. Pat. No. 4,741,326), whose disclosures are incorporated herein by reference. These methods attempt to prevent contamination of the endoscope, either by adding disposable working channels external to the endoscope itself (Silverstein) or by adding a disposable liner inside a working channel of the endoscope (Sidall).

Voloshin (U.S. Pat. No. 6,485,409), whose disclosure is incorporated herein by reference, describes an endoscope, which comprises an endoscopic probe, a bending section for directing the probe within the colon (steering unit), an insertion tube and a flexible covering sleeve or a sheath, which is coupled proximally to the probe. The sleeve is attached to the endoscope in such a manner that its folded section is retained between a cap and an internal spindle located between the insertion tube and the probe head. When inflated, the folded section unfolds over a flange of the internal spindle and an inner portion of the sleeve is pulled in a distal direction. The sleeve at the same time covers the insertion tube and prevents its contamination during the endoscopic procedure.

Eizenfeld (WO 2004/016299; PCT/IL03/000661), whose disclosure is incorporated herein by reference, discloses an endoscope which employs a flexible inflatable sleeve assisting propulsion of the insertion tube within the body organ. The sleeve is retained in folded condition within a dedicated dispenser. The insertion tube is inserted into a dispenser and is advanced within the body organ. The insertion tube engages the sleeve, which covers the insertion tube and protects it from contamination.

Bar-Or (WO 2005/110185; PCT/IL05/000426), whose disclosure is incorporated herein by reference, discloses a disposable set which comprises a dispenser for retaining a folded disposable sleeve, which upon inflation unfolds and protects the insertion tube from contamination.

The above-mentioned references teach how the principle of a disposable covering sleeve can be realized essentially in a colonoscopic apparatus, however they do not disclose how to implement this approach for preventing contamination in a gastroscopic apparatus as well.

### SUMMARY OF THE INVENTION

The object of the present invention is to provide a new and improved gastroscopic apparatus, which minimizes the risk of cross contamination to both patient and staff during the gastroscopic procedure.

Still a further object of the invention is to provide a new and improved gastroscopic apparatus, wherein an insertion tube can be advanced along the esophagus while being protected by a disposable covering sleeve.

Still a further object of the invention is to provide a new and improved gastroscopic apparatus fitted with a simple arrangement for deployment of a covering sleeve around the insertion tube when it is being inserted in the esophagus and displaced along the esophagus.

Various embodiments of the present invention can be implemented as a gastroscopic apparatus and as a method for propelling a gastroscope in the esophagus.

For a better understanding of the present invention as well of its benefits and advantages, reference will now be made to the following description of its embodiments, taken in combination with the accompanying drawings.

### BRIEF DESCRIPTION OF THE DRAWINGS

FIG. 1 is a perspective view of an operation handle of a gastroscopic apparatus of the present invention.

FIG. 2 is a longitudinal view, partially broken away, of a fragment of the insertion tube coupled to a dispenser.

FIG. 3A is a longitudinal view, partially broken away, of the dispenser just before entry into the mouthpiece; and, FIG. 3B is an enlarged sectional view of area A of the mouthpiece.

FIG. 3C is a longitudinal view, partially broken away, of the dispenser upon entry into the mouthpiece and arresting of the dispenser within the mouthpiece; and, FIG. 3D is an enlarged sectional view of area A of the mouthpiece encircled in FIG. 3C.

FIG. 4 is an isometric view of a mouthpiece employed in the gastroscopic apparatus of the present invention.

FIG. 5 shows the insertion tube before insertion into the mouthpiece.

FIG. 6 shows the insertion tube inserted into the mouthpiece and at the beginning of the gastroscopic procedure.

FIG. 7 shows the insertion tube at the beginning of the advancement through the mouthpiece into the esophagus and FIG. 8 shows the entire gastroscopic apparatus and the patient during the gastroscopic procedure.

### DETAILED DESCRIPTION OF THE INVENTION

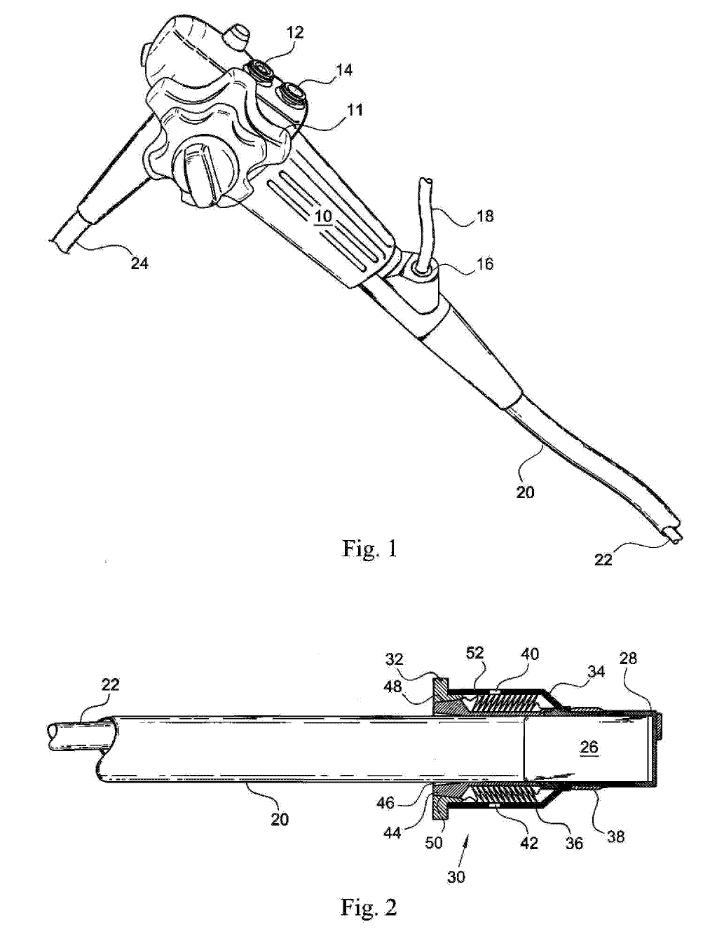

With reference to FIG. 1 an operation handle 10 of a gastroscopic apparatus of the present invention is shown. The operation handle is of a conventional design and is identical with the operation handle of a standard gastroscopic apparatus in all its functionality for the operator. The gastroscopic apparatus comprises also a system control unit (SCU) and a monitor. Since these components are similar with those of the standard gastroscopic apparatus they are not shown in FIG. 1, but are seen in FIG. 8.

The operation handle includes an angulation control knob or wheel 11, suction and air/water buttons 12, 14 for admitting fluid medium into the esophagus and a Y-connector 16 provided with an inlet port 18 and a channel for connecting to respective sources of fluid medium (usually air, water and vacuum). These sources are available at the SCU.

The operation handle is operatively connected to a proximal end of an insertion tube 20, through which a guide channel 22 extends. In a preferred embodiment of the invention the guide channel is designed as an integral conduit suitable for receiving a disposable tube with separate lumens for supplying air, water and vacuum. This tube will be referred-to further as multilumen tubing. An example of such multilumen tubing and its description can be found in Bar-Or (WO 2005/110200; PCT/IL05/000428), whose disclosure is incorporated herein by reference. The operation handle is connected also to a system control unit (SCU, shown in FIG. 8) via an umbilical cord 24, through which extend electrical cables connecting the SCU with the optical head deployed in the distal end of the insertion tube. All above elements are similar to those employed in the conventional gastroscopic apparatuses.

It is not shown in detail, but one should keep in mind that within the insertion tube are provided various devices, which are necessary for proper functioning of the endoscope. These devices and their arrangement within the insertion tube are known in the art. Among such devices one can mention vertebrae and strings, which can be manipulated by the angulation knob provided at the operation handle.

Referring now to FIG. 2 it is shown that at the distal end of the insertion tube 20 an optical head 26 covered by a plastic cap 28 is provided. The plastic cap is defined by a forward butt end and by a cylindrical periphery surface. The butt end is conveniently provided with windows, which are aligned with an optical camera located within the optical head and with a light source of the optical camera. In accordance with a preferred embodiment a CCD camera should be used. The optical head is fitted also with an integrated light source, preferably white LED light source. By virtue of the CCD camera and the LED light source, visualization is much more efficient since there is no need for fiber optics, and thus it is possible to significantly reduce maintenance and repair costs. An example of a suitable optical head provided with the CCD camera and the LED light source can be found in our patent application PCT/IL05/000929 whose disclosure is incorporated herein by reference.

One should also keep in mind that adjacent the distal section of the insertion tube a bending section is provided. This section enables maneuvering of the distal end of the insertion tube during the endoscopic procedure. The bending section comprises a plurality of vertebrae connected to the strings, which are pullable upon rotation of the angulation knob. By virtue of this provision it is possible to change the vertebrae's position and thus to control the curvature of the distal end and to navigate the insertion tube during its advancement within the patient.

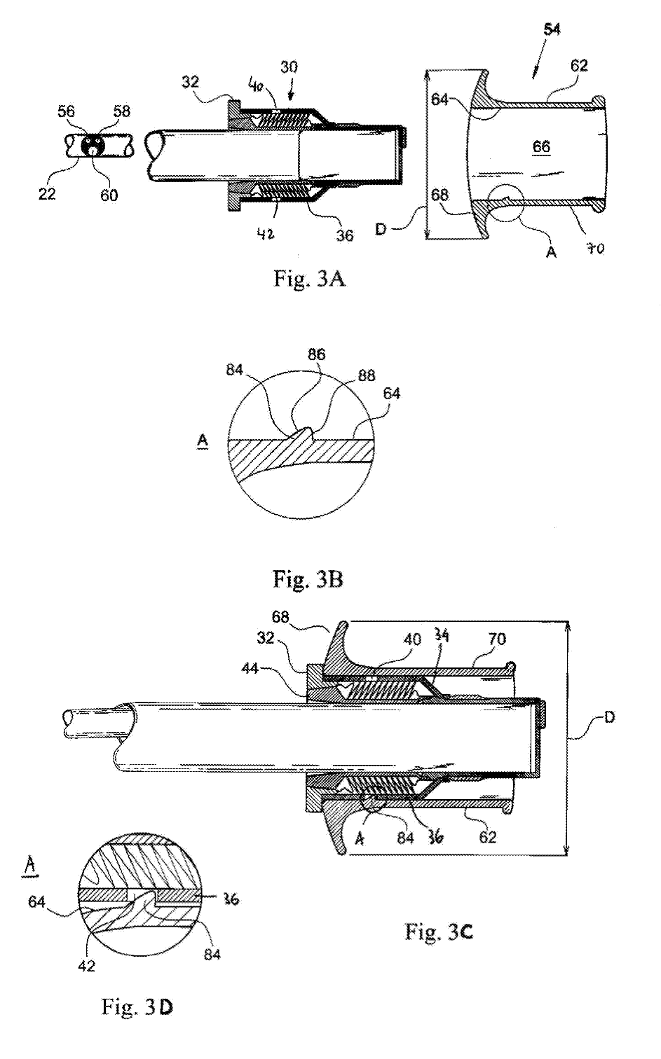

FIG. 2 shows still a further component of the gastroscopic apparatus of the invention. This component is a dispenser 30, which is provided at the distal end of the insertion tube and is used during the gastroscopic procedure. The dispenser is configured as a tubular body through which the insertion tube can pass. The dispenser has a rear end 32, an elastically flexible forward end 34 and an intermediate portion 36 confined therebetween. At the beginning of the gastroscopic procedure the dispenser is coupled to the insertion tube in such a manner that its rear end 32 is located at the bending section and its forward end 34 elastically embraces the cap. The dispenser is coupled to the insertion tube at the forward end with a possibility that during the gastroscopic procedure the insertion tube can be protracted into or retraced from the dispenser. Tightly put on the cap a snap ring 38 is provided, which allows detachable snap connection between the forward end 34 and the insertion tube. The snap connection at the same time ensures that the insertion tube would be only distally displaceable with respect to the dispenser. The intermediate portion of the dispenser is formed with a couple of diametrically disposed through going openings 40, 42, which function will be explained later on with reference to FIGS. 3C and 3D.

Deployed within the dispenser a fixation bushing 44 is provided. This bushing has a flaring entrance opening 46 to pass the insertion tube there through and a conical outside surface 48, which snugly fits with a mating inwardly facing conical surface 50 at the rear end of the dispenser. The remainder of the bushing is configured as a thin cylindrical tube, which is put on the insertion tube with possibility for relative displacement there between.

An annular space is defined between intermediate portion 36 of the dispenser and the bushing and a flexible protection sleeve or sheath 52 is deployed in this space. The sleeve is folded within the dispenser as shown in FIG. 2, while its proximal end is firmly anchored between conical surfaces 48,50. The sleeve's distal end passes between the cap and the forward end 34 and is firmly anchored between the cap's periphery and the snap ring.

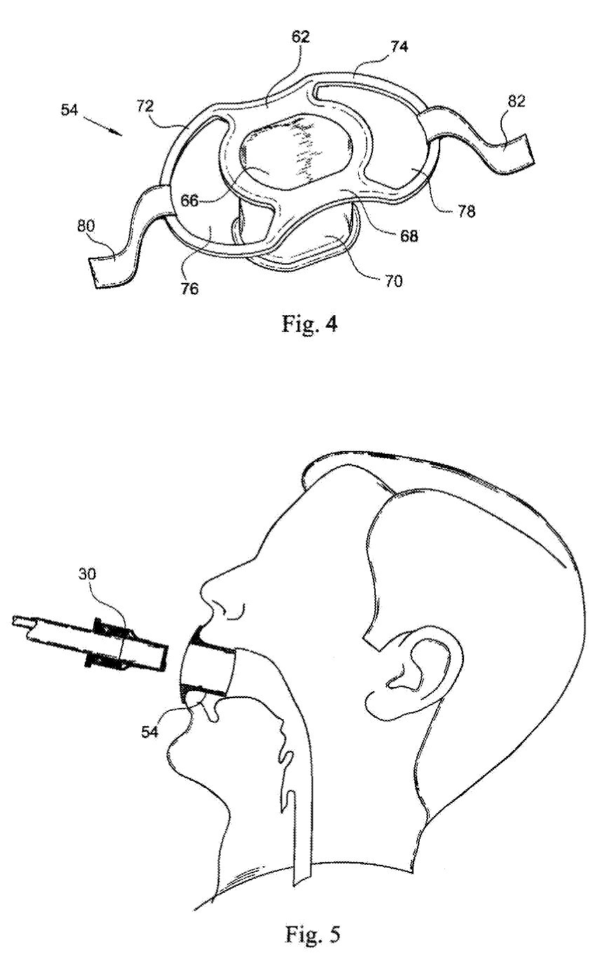

Now with reference to FIGS. 3A, 3B, 3C, 3D and 4 still a further component of the gastroscopic apparatus of the invention will be described. This component is a mouthpiece 54 depicted in FIGS. 3A and 4.

During the gastroscopic procedure the mouthpiece is held within the patient's mouth by her or his teeth and the insertion tube is advanced along the esophagus through the mouthpiece, while the dispenser is arrested within the mouthpiece.

FIG. 3A also schematically shows the cross-section of the integral guide channel or multilumen tubing with separate lumens 56, 58, 60 extending there along. Lumens 56,58 are of a smaller diameter and they are respectively intended for supplying water for irrigation and air for insufflation. Lumen 60 is of a larger diameter and is intended for introducing surgical tools or for suction.

As shown in FIG. 3A and 3B the mouthpiece is provided with a central body portion 62, which has a tubular shape. An inwardly facing cylindrical surface 64 of the central body portion defines an opening 66 through which the dispenser can be brought in the mouthpiece. To allow this, the inside diameter of the opening slightly exceeds the outside diameter of the intermediate portion 36 of the dispenser. As will be explained later on, by virtue of this provision the dispenser also can be rotated along its longitudinal axis when it is brought within the mouthpiece.

One end of the central body portion of the mouthpiece terminates by a flange portion 68, while the opposite end terminates by a distal, bite portion 70. The flange portion is directed essentially perpendicularly to the central body portion and has an overall dimension D. The inside diameter of the opening 66 is less than the outside diameter of the rear end 32 of the dispenser. By virtue of this provision when the dispenser is fully inserted within the mouthpiece, the flange portion 68 of the mouthpiece abuts the rear end 32 of the dispenser to limit its axial displacement within the mouthpiece as seen in FIG. 3C.

The bite portion is configured with a rim, which slightly widens laterally to ensure reliable holding of the bite portion by the patient's teeth when the mouthpiece is in the patient's mouth.

FIG. 4 shows the flange portion 68 of the mouthpiece in more detail. One can see that it has nearly elliptical configuration defined by two rounded wings 72, 74. The wings are situated diametrically at both sides of the central body portion 62. To reduce weight of the mouthpiece, respective openings 76, 78 are provided within the rounded wings. It is seen also that respective ends 80, 82 of an elastic strap are attached to each wing. During the gastroscopic procedure the strap is worn behind the back of patient's head and holds the mouthpiece more reliably in place within the patient's mouth.

Attention is now called again to FIGS. 3A, 3B, 3C and 3D. These figures show that a protrusion 84 is formed on the inwardly facing surface 64 of the mouthpiece. The protrusion is made of elastically deformable material and has a saw tooth shape defined by a long inclined surface 86 and by a short inclined surface 88. The inclination angle of the surface 86 is less than inclination angle of the surfaces 88. The surface 86 is inclined to allow advancement of the dispenser within the mouthpiece only in the distal direction. Dispenser 30 is allowed to advance along the mouthpiece until protrusion 84 enters in one of the through-going opening 40 or 42, depending on the relative angular disposition of the dispenser within the mouthpiece. As soon as protrusion 84 is aligned with one of the openings, it elastically snaps and the dispenser becomes arrested within the mouthpiece. This situation is shown in FIGS. 3C and 3D. To separate the dispenser from the mouthpiece one should rotate the dispenser so as to elastically deform the protrusion and to remove it from the opening. Rigidity of dispenser material as well as configuration and dimension of the protrusion are selected to allow elastic deformation of the protrusion resulting in snapping engagement and disengagement between protrusion and openings depending whether dispenser is advanced within the mouthpiece, or rotated.

In practice the dispenser, the sleeve and the mouthpiece are cheap disposable items, which are discarded at the end of the endoscopic procedure after evacuating the insertion tube from the esophagus. The dispenser and the mouthpiece could be made from a rigid or semi rigid plastic material, e.g. polypropylene, polyethylene, ABS, etc.

The sleeve could be typically made from a flexible biocompatible plastic, such as polyamide, having a thickness of about several tens of microns.

In FIGS. 5-8 are shown the consecutive stages of a gastroscopic procedure during which the gastroscopic apparatus of the present invention is used in practice.

FIG. 5 presents an initial stage of the gastroscopic procedure when the mouthpiece 54 is held within the patient's mouth being ready to receive the distal end of the insertion tube along with the dispenser 30 coupled thereto. For the sake of simplicity the other components of the gastroscopic apparatus are not shown.

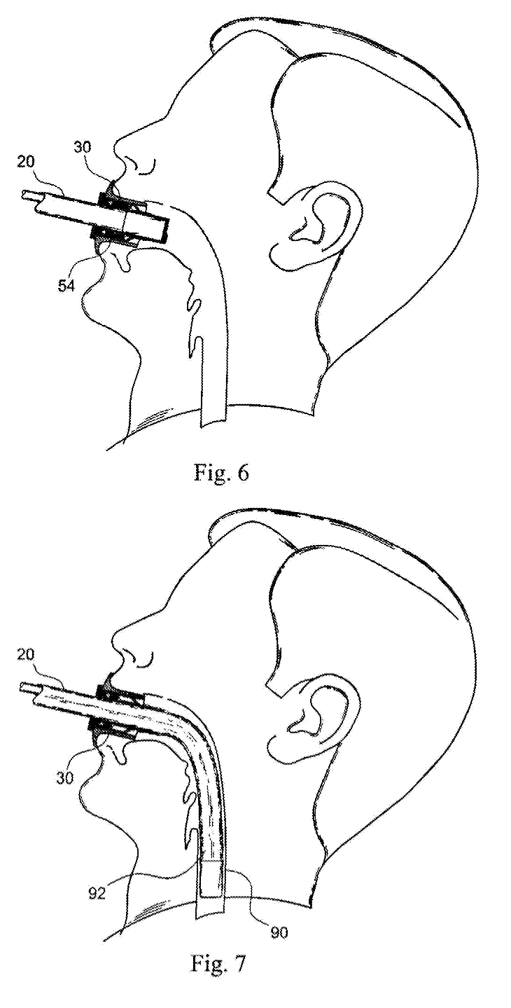

FIG. 6 shows the next stage of the gastroscopic procedure, during which the distal end of the insertion tube has been brought towards the opening of the mouthpiece and then advanced there into. As explained earlier by rotating the dispenser within the mouthpiece one of the openings 40, 42 can be brought in alignment with the protrusion 84 to allow snapping thereof on the intermediate portion 36 of the dispenser and arresting within the mouthpiece.

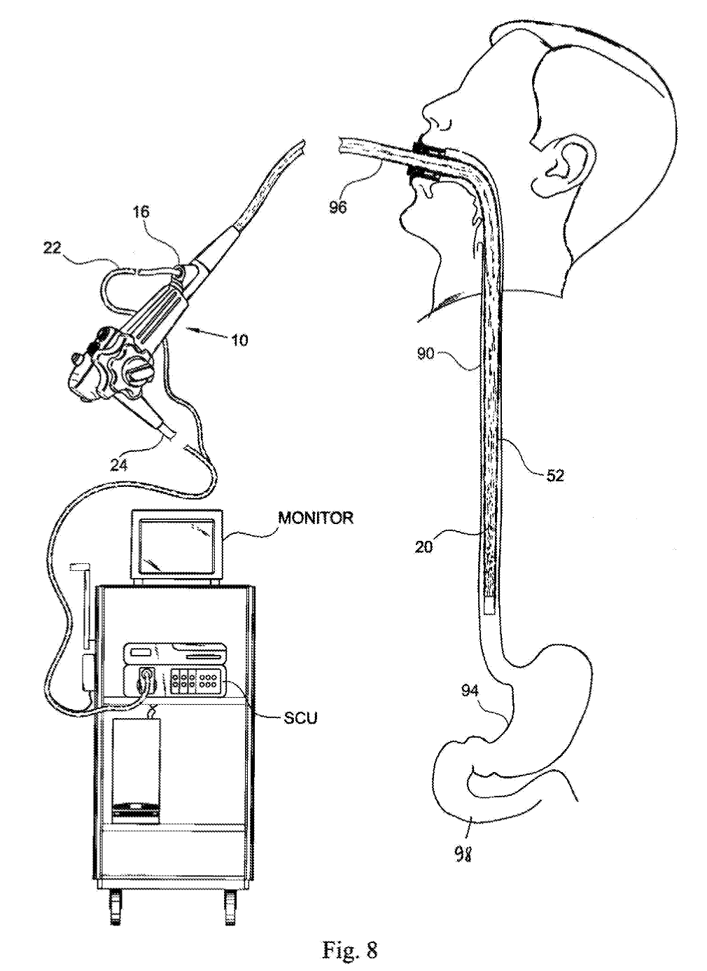

Still further stage of the gastroscopic procedure is depicted in FIG. 7, showing how the insertion tube has been pushed further through the dispenser 30. The dispenser is arrested within the mouthpiece and remains stationary. The proximal end of the sleeve remains stationary too, since it is firmly secured at the rear end of the dispenser. Insertion tube 20 is pushed distally through the dispenser along the esophagus 90 and urges the distal end of the sleeve to move distally together with the insertion tube, since it is firmly secured between the cap and the insertion tube. Distal end of the sleeve extends automatically from the dispenser, unfolds and covers a portion 92 of the insertion tube, which has passed through the dispenser. It can be appreciated that the unfolded sleeve deploys around the insertion tube and reliably protects it from any contamination matter originating from the esophagus.

FIG. 8 depicts a still further stage of the advancement of the insertion tube. It is seen that at this stage a major portion of the insertion tube has passed the dispenser and is located within the esophagus. This portion is covered by protective sleeve 52. Distal end of the insertion tube is approaching the stomach 94, which interior can be observed by the optical head. Other components of the gastroscopic apparatus remain outside the patient. Among these components are proximal portion 96 of the insertion tube 20 and operation handle 10 with Y-connector 16, working channel 22, umbilical cord 24, the SCU and the monitor.

It is seen that proximal portion 96 of the insertion tube still has not been advanced in the dispenser. This portion would be available if it is required to advance the insertion tube along the esophagus still further, through the stomach 94 within a duodenum 98.

At the end of gastroscopic procedure the insertion tube and the sleeve are withdrawn from the patient.

According to one embodiment of the invention the dispenser remains arrested within the mouthpiece during the withdrawal. When the insertion tube is being withdrawn, the sleeve bunches and remains in the patient's mouth outside the dispenser. At the end of withdrawal the compactly bunched sleeve is located in the mouth adjacent the bite portion. Now the dispenser is disengaged from the mouthpiece and the insertion tube together with the bunched sleeve is taken out. Thereafter the insertion tube is separated from the sleeve and the sleeve along with the dispenser is disposed of. The mouthpiece is removed from the patient's mouth and is disposed of too.

According to an alternative embodiment the dispenser is disengaged from the mouthpiece before the withdrawal and the insertion tube is evacuated from the esophagus together with the sleeve being extended and deployed there along. The insertion tube is removed from the mouthpiece together with the dispenser and with the covering sleeve. Thereafter the insertion tube is separated from the sleeve and the sleeve along with the dispenser is disposed of. The mouthpiece is removed from the patient's mouth and is disposed of too.

It can be readily appreciated that by virtue of the present invention it is possible to minimize the risk of cross contaminations to both patients and staff during the gastroscopic procedure and to make the gastroscopic procedure cleaner and safer.

Furthermore, the present invention eliminates the need for reprocessing equipment and helps the facility to save on capital expenses (purchase of additional scopes), labor costs and costs of disinfectants, cleaning tools and scope maintenance.

The present invention allows for increased patient throughput as a result of faster procedures with shorter down time for the scope. This, in its turn, allows to schedule more procedures and therefore to increase revenues for both physician and facility.

The gastroscopic apparatus of the present invention has very simple construction, which is reliable and at the same time remains very similar to a standard apparatus, thus enabling an extremely short learning time to the physician to achieve optimal performance.

It should be appreciated that the present invention is not limited to the above-described embodiments and that one ordinarily skilled in the art can make modifications without deviation from the scope of the invention, as will be defined in the appended claims.

For example, more that two openings could be made in the dispenser and more than one tooth could be made in the mouthpiece.

The openings could be made in the mouthpiece instead of the dispenser and the teeth can be arranged in the dispenser instead of the mouthpiece.

Other types of detachable connections between the mouthpiece and the dispenser could be employed instead of a snap connection.

When used in the following claims, the meaning of terms “comprise”, “include”, “have” and their conjugates is “including but not limited to”.

It should also be appreciated that the features disclosed in the foregoing description, and/or in the following claims, and/or in the accompanying drawings may, both separately and in any combination thereof, be material for realizing the present invention in diverse forms thereof.

We claim:

1. A gastroscopic apparatus for inspecting a body channel, said apparatus comprising
an insertion tube provided at a distal end thereof with an optical head for visualization the body channel,
an operating handle with a means for navigating the distal end of the insertion tube during advancement thereof along the body channel and with a means for controlling the supply of a fluid medium to the body channel,
a dispenser detachably coupled to the insertion tube, said dispenser is provided with a rear end, a forward end and an intermediate portion confined there between, said dispenser is configured as a tubular body through which said insertion tube can be protracted or retracted,
a covering sleeve retained within the dispenser,
a mouthpiece through which the dispenser can be brought in the body channel, wherein said covering sleeve is deployable on the insertion tube during displacement of the insertion tube along the body channel.

2. The gastroscopic apparatus as defined in claim 1, wherein said insertion tube being relatively displaceable with respect to said dispenser.

3. The gastroscopic apparatus as defined in claim 2, wherein said covering sleeve being retained within the dispenser in a folded state, wherein a proximal end of said sleeve being secured at the dispenser and a distal end of said sleeve being secured at the insertion tube.

4. The gastroscopic apparatus as defined in claim 3, wherein said dispenser being releasably arrestable within the mouthpiece.

5. The gastroscopic apparatus as defined in claim 4, wherein the distal end of the covering sleeve being distally extendable from the dispenser upon advancing the insertion tube.

6. The gastroscopic apparatus as defined in claim 5, wherein said dispenser being relatively displaceable with respect to the mouthpiece.

7. The gastroscopic apparatus as defined in claim 6, wherein said dispenser being linearly displaceable with respect to the mouthpiece.

8. The gastroscopic apparatus as defined in claim 6, wherein said dispenser being rotatable with respect to the mouthpiece.

9. The gastroscopic apparatus as defined in claim 1, wherein said dispenser is arrestable in the mouthpiece by a snap connection.

10. The gastroscopic apparatus as defined in claim 9, wherein said snap connection includes at least one protrusion receivable within a corresponding at least one opening.

11. The gastroscopic apparatus as defined in claim 10, wherein said at least one protrusion being made in the mouthpiece and said at least one opening being made in the dispenser.

12. The gastroscopic apparatus as defined in claim 10, further comprising one protrusion and two diametrically disposed openings made in the dispenser.

13. The gastroscopic apparatus as defined in claim 12, further comprising a cap covering a distal end of the insertion tube, said cap being embraced by the forward end of the dispenser.

14. The gastroscopic apparatus as defined in claim 13, further comprising a snap ring tightly put on the cap.

15. The gastroscopic apparatus as defined in claim 3, wherein the distal end of the covering sleeve being secured between the cap and the snap ring.

16. The gastroscopic apparatus as defined in claim 1, wherein said body channel being an esophagus.

17. A method for carrying out of a gastroscopic procedure during which an insertion tube of a gastroscopic apparatus is advanced within a body channel of a patient, said method comprises
providing a dispenser detachably coupled to the insertion tube, said dispenser retaining a covering sleeve,
providing a mouthpiece holdable within a mouth of said patient,
bringing said dispenser together with the covering sleeve into the mouthpiece,
arresting said dispenser within the mouthpiece,
advancing the insertion tube within the body channel through the dispenser to cause said covering sleeve to extend from the dispenser and to deploy around the insertion tube,
visualization of the body channel,
performing, if necessary, a therapeutic procedure,
withdrawing of the insertion tube from the body channel.

18. The method as defined in claim 17, wherein said covering sleeve being retained within said dispenser in a folded state while upon extension from the dispenser said sleeve unfolds and covers a portion of the insertion tube which had passed through the dispenser.

19. The method as defined in claim 17, wherein said withdrawing comprises detaching of said dispenser from the mouthpiece and evacuating the dispenser, the insertion tube and the sleeve from the body channel, wherein during evacuation said sleeve remaining unfolded and deployed along the insertion tube.

20. The method as defined in claim 19, wherein said withdrawing of the insertion tube comprises retracting of the insertion tube from the body channel accompanied by bunching of the covering sleeve proximate to the mouthpiece, detaching of said dispenser from the mouthpiece and simultaneous evacuation of the dispenser, the insertion tube and the bunched sleeve from the body channel.

21. A mouthpiece for holding within a patient mouth and for carrying a dispenser with retained therein a sleeve for covering an insertion tube of a gastroscopic apparatus, said mouthpiece comprises
a central body portion defining an opening through which the dispenser can be brought in the mouthpiece, said central body portion is confined between a flange portion and a bite portion; and
a means for detachable arresting the dispenser in the mouthpiece, wherein upon arresting the dispenser and displacement of the insertion tube said sleeve is extendable from the dispenser and deployable on the insertion tube.
